(19) United States
(12) Patent Application Publication
(10) Pub. No.: US 2024/0248083 A1
(43) Pub. Date: Jul. 25, 2024

(54) SALIVA PAIRED IMAGING TEST APPARATUS AND METHOD

(71) Applicant: Oral Genome Corp., Carlsbad, CA (US)

(72) Inventor: Tina Saw, Carlsbad, CA (US)

(73) Assignee: Oral Genome Corp., Carlsbad, CA (US)

(21) Appl. No.: 18/422,976

(22) Filed: Jan. 25, 2024

Related U.S. Application Data
(60) Provisional application No. 63/441,041, filed on Jan. 25, 2023.

(51) Int. Cl. G01N 33/543; G01N 21/77; G01N 21/78; G01N 33/569; G01N 33/573

(52) U.S. Cl. CPC G01N 33/54388; G01N 21/78; G01N 33/56911; G01N 33/56955; G01N 33/573; G01N 2021/7759; G01N 2333/91091; G01N 2333/96433; G01N 2470/04

(57) ABSTRACT

An apparatus and method for remote saliva testing in a variety of environments. The test involves the healthcare provider and the patient. Included is a non-invasive method of saliva collection and result detection which allows healthcare providers to send test kits to patient homes for remote testing, monitoring and care decisions. The apparatus includes a dental saliva imaging test card paired with a web application downloaded to a smartphone. The system may be used at home or in the dental office. The smartphone interface walks patients and providers through the test with tutorials to accurately capture testing results. The imaging test card includes a plurality of test strips onto which the patient places saliva. The test strips are sensitized to look for certain biomarkers, some of which detect periodontal disease and elevated caries risk. Results from the test strips are photographed and are then uploaded to the application for analysis.

### RELATED APPLICATION

This application claims priority to U.S. Provisional Patent Application No. 63/441,041, titled “SALIVA PAIRED IMAGING TEST SYSTEM AND APPARATUS,” filed on Jan. 25, 2023, the entire contents of which are incorporated by reference herein.

### TECHNICAL FIELD

The present invention relates to the field of dental saliva testing. More particularly, the disclosed inventive concept relates to a dental saliva test cassette or card which may be used in conjunction with Applicant's application on a tablet configured for use in either a dental clinic or in the patient's home which allows patients and providers to easily test their saliva in real time.

### BACKGROUND OF THE INVENTION

Nearly 3.5 billion people worldwide are affected by oral disease, and oral diseases, while largely preventable, pose a major health burden for many countries and affect people throughout their lifetime, causing pain, discomfort, disfigurement and even death [World Health Organization, 2022]. Untreated dental caries is the most common health condition [Global Burden of Disease, 2019]. Oral diseases are caused by a range of modifiable risk factors, yet 40% of adults will experience oral pain yearly and more than 80% of people will have at least one cavity by age 34 [Centers for Disease Control, 2022]. Proactive approaches in dentistry prevents problems from happening, identifies oral diseases and corrects them early on. Reactive approaches in dentistry patches carious holes and reduce inflammation, but does not correct for the underlying cause. In medicine, blood tests are routine to check for underlying diseases and conditions, allowing for early prevention. In dentistry, routine x-rays and clinical exams are performed to see and treat damage that has already occurred. 94% of all diseases start in the mouth, financially impacting the United States' healthcare system [Cigna Health Insurance, 2019]. An alternative approach, chairside salivary analysis through test cards promotes preventive and early treatment modalities for providers and personalized at-home prevention plans for patients. According to the inventive apparatus and method disclosed herein, Applicant provides a chairside salivary analysis test card paired with computer vision to allow for quick and easy-to-understand reports about dental health and oral-systemic conditions for patients and providers, along with disease progression tracking.

Earlier efforts in salivary testing focused on ELISA-based assays (Enzyme-Linked ImmunoSorbent Assay) that require a provider to collect the saliva sample in-office, put in a stabilizing solution and mail the sample back. However, due to the limited functionality and preservation capabilities of the saliva stabilizers, as well as the high cost and time for mailing and processing, salivary testing has not been routinely adopted as an early detection and prevention method for patients. Applicant's studies showed that the stability of saliva does not hold up over time. In fact, degradation of tested saliva was found to begin as soon as two hours following isolation. Accordingly, point-of-care testing is critically important because the purest form of the saliva is immediately studied and evaluated before degradation begins. Another disadvantage of the traditional approach to evaluation is that most saliva tests have a high run cost with the average being $550 per traditional ELISA assay [Bio-Techne, 2022], and providers must bring patients in for another visit to review results with patients and better modify treatment modalities. This is not conducive to a normal dental office workflow.

In recent years, advances in artificial intelligence, machine learning and computer vision in dentistry help providers more accurately diagnose and provide better treatment recommendations. Related techniques have been applied to radiograph related systems, such as Pearl AI and Velmeni™. These technologies assist providers in better predictive, more reliable and diagnostic intelligence. However, these technologies can only assist in measuring and detecting the extent of conditions that already occurred, not how to minimize or correct conditions early on.

### SUMMARY OF THE INVENTION

The disclosed inventive composition provides a method and apparatus for saliva testing in a variety of environments. The test involves the healthcare provider and patient. Healthcare providers can include dentists, dental hygienists, dental assistants, physicians, physician assistants, nurses and nursing assistants. These healthcare providers can be in dental offices, nursing homes, community clinics and more. The easy, non-invasive methods of saliva collection and result detection set forth herein also allow providers to send test kits to patient homes for remote testing, monitoring and care decisions.

Applicant Oral Genome® (OG) corporation developed a Saliva Paired Imaging Test™ (SPIT) that provides dental saliva testing in a more effective, time-efficient and lower cost manner than any previously available solution to all populations. Applicant's dental saliva card paired with a robust web application on a touch-screen Android® or smartphone tablet configured for use in either a dental clinic or home allows patients and providers to easily test their saliva. The interface walks patients and providers through the test with easy step-by-step tutorials to accurately capture testing results and recommendations (FIGS. 1A and 1B).

Applicant Oral Genome® completed assay feasibility for lateral flow assays and colorimetric assays for the following biomarkers: (1) MMP-8: an inflammation marker that is elevated in saliva when patients exhibit signs of periodontal disease, (2) Porphyromonas gingivalis: a bacteria elevated in patients who have periodontal disease, (3) proteins, (4) nitric oxide (N2O), (5) salivary pH: decreased in those with elevated caries risk, (7) buffering capacity: decreased in those with elevated caries risk and (7) glucose. Applicant identified an additional marker, Glucosyltransferase B: an enzyme produced by Streptocococcus mutans and other caries causing bacteria, which catalyzes synthesis from sucrose of insoluble glucans to provide support to the biofilm such as dental plaque. Commercial antibodies were initially screened through PCR to ensure detection. Limit of detection studies were also completed for MMP-8 (FIG. 3) and P. gingivalis (FIG. 5), and replication was ensured (FIGS. 4 and 6).

Cross reactivity with other common oral bacteria such as Streptococcus mutans, Fusobacterium nucleatum and Lacticaseibacillus rhamnosus was also studied, along with microbial interferences. No cross reactivity or microbial interferences were found (FIGS. 7A, 7B, 8A and 8B). A high-dose hook effect, which is the state of antigen excess relative to the antibody probes resulting in falsely lowered values, was also studied, and no high dose hook effect was found, ensuring no false results (FIGS. 9 and 10). Common endogenous and exogenous interferences in saliva, such as alcohol, toothpaste, Nyquil®, mouthwash and genomic DNA were also studied and no interferences were found (FIGS. 11 and 12).

Applicant's feasibility studies confirmed that detection through lateral flow assays and colorimetric assays for MMP-8 and P. gingivalis are feasible and easily detectable with a human eye. Salivary pH, buffering capacity and ammonia tests currently exist in medicine through colorimetric assays, and can easily be added to the test card and allow providers and patients to make optimal home care and treatment decisions. Using antibodies for Glucosyltransferase B and applying the same techniques and methods allows optimization of the biomarker on a lateral flow assay (LFA) and ultimately on a test card.

### Evidence of Oral-Systemic Health and Current Detection Methods

The oral cavity is the intersection of medicine and dentistry, and is the window into the general health of a patient. Oral health is a key indicator of overall health, well-being and quality of life [World Health Organization, 2021]. Oral diseases vary in forms from dental caries, periodontal disease, cancer, Sjögren's Syndrome, halitosis, candida and more. Many of these diseases require extensive treatment and are directly linked to how the body functions, the development of specific diseases and even mortality. Oral infections travel through the alimentary tract as bacteria is swallowed. Many of these bacteria survive the acidic environment of the stomach and get transmitted through the bloodstream or lymphatics to cause atherosclerosis, diabetes, endocarditis, preterm birth and other systemic diseases [Oral Infections and Systemic Diseases, 2022].

When systemic diseases have oral symptoms such as ulcerations, salivary gland swelling, bone lesions and tongue afflictions, it is easy for dentists to visualize and refer the patient to medical doctors for further testing and appropriate treatment. The same applies for oral symptoms such as visible openings, holes and discolorations of teeth and bleeding gums. When a dentist visualizes these symptoms, and verifies through a radiograph, they can appropriately treat the tooth with a filling, crown, root canal and more depending on the extent of the lesion.

Currently, clinical exams are paired with imaging equipment such as radiographs and intraoral cameras. Clinical exams and imaging methods detect disease that has already occurred and caused destruction. It takes over 60% of bone loss to happen until dentists see it on x-rays, forcing dentists to react versus prevent.

Biomarker-based technologies have been used for the identification of oral diseases and risk levels. However, all these technologies require time due to mailing and processing. Real-time and chairside biomarker-based technologies for the identification of Sjögren's Syndrome. Imaging diagnostics to accelerate clinical implementation of reliable, reproducible, highly specific and sensitive diagnostic instruments for dental caries, periodontal disease, halitosis and candida do not exist. Furthermore, there is no salivary detection to indicate control of diabetes, which if uncontrolled can lead to secondary infections such as candidiasis, parotid salivary gland enlargement, gingivitis, periodontitis, xerostomia and burning mouth sensations [American Dental Association, 2022].

Applicant Oral Genome® relied on its previous preliminary feasibility studies to develop improved methods to detect and predict progression of dental caries, periodontal disease, Sjögren's Syndrome, halitosis, candida and diabetes.

### Healthcare Advancements Provided by the Present Apparatus and Method

Presently, dental health professionals diagnose and treat based on clinical exams and radiographs. Periodontal probings and gum tissue pocket measurements are completed by dentists and hygienists, along with recession and bleeding points to assist in the diagnosis and treatment recommendations for periodontal disease. Probing and measurements can vary by provider, and determination of carious lesions and halitosis can also vary and be subjective. Early detection and diagnosis can be challenging, and providers take yearly x-rays to watch the progression of dental diseases. The disease progression varies by patient depending on medication, salivary pH, buffering capacity, diet, lifestyle habits and more. in response to this need, Applicant designed and developed an objective saliva paired imaging test (SPIT) in which computer vision can analyze and detect biomarker levels in real-time, while outputting immediate results and recommendations for better patient home care and objective and accurate provider diagnosis. The computer vision is integrated with a Web application requiring only a minimal amount of human intervention in the detection of reliable results in a minimum amount of time, thereby utilizing stable salivary conditions.

In recent years, computer vision and AI-powered healthcare testing have rapidly developed. Real-time detection products in medicine for COVID-19 testing, pregnancy testing and vitamin and mineral testing utilizing lateral flow assays and colorimetric assays paired with computer vision and AI have been proven to be commercially successful. However, real-time salivary testing in dentistry has not been integrated due to lack of technologies with dental biomarkers, high costs and non-comprehensive panel of markers to make sound clinical diagnosis and treatment plans. Applicant's well-defined use case and proprietary feasibility data provided improved technology which is applicable to dentistry. Applicant's SPIT test not only assists dentists with diagnosis and treatment decisions, but also serves all patients, including underserved populations and environments where there are provider shortages and remote monitoring and coaching are critical to achieve healthier oral-systemic health. Not only does the apparatus and method work directly to permit cost-effective and efficient apparatus production and optimized computer vision detection around the SPIT test, but also the disclosed apparatus and method may be delivered to underserved communities in conjunction with AWS' Health Equity's Global Initiative and community partners to help increase oral health literacy, remotely monitor and coach patients and reduce long-term oral-systemic impacts.

### DESCRIPTION OF THE DRAWINGS

The patient or application contains at least one drawing executed in color. Copies of this patient or patient application publication with color drawing(s) will be provided by the Office upon request and payment of the necessary fee. For a more complete understanding of this invention, reference should now be made to the accompanying figures in which:

FIGS. 1A and 1B Illustrates an interface showing how patients and/or providers input information;

FIG. 2 illustrates a SPIT test card design having multiple separated LFA lanes and colorimetric spaces;

FIG. 3 illustrates limit of detection studies for MMP-8;

FIG. 4 illustrates limit of detection studies for P. gingivalis;

FIG. 5 also illustrates limit of detection studies for P. gingivalis;

FIG. 6 illustrates additional limit of detection studies for P. gingivalis;

FIGS. 7A and 7B illustrate cross-reactivity and microbial interference studies on LFA against S. mutans, P. gingivalis, L. rhamnosus, S. oralis and F. nucleatum confirm no cross reactivity for MMP-8;

FIGS. 8A and 8B illustrate cross-reactivity and microbial interference studies on LFA against S. mutans, P. gingivalis, L. rhamnosus, S. oralis and F. nucleatum confirm no cross reactivity for P. gingivalis;

FIG. 9 illustrates a MMP-8 validation study on LFA to confirm for no high dose hook effect;

FIG. 10 illustrates a P. gingivalis validation study on LFA to confirm for no high dose hook effect;

FIG. 11 illustrates a MMP-8 validation study on LFA to confirm no interferences against alcohol, toothpaste, Nyquil®, mouthwash and genomic DNA in saliva;

FIG. 12 illustrates a P. gingivalis validation study on LFA to confirm no interferences against alcohol, toothpaste, Nyquil®, mouthwash and genomic DNA in saliva; and

FIG. 13 illustrates results shown on a computer screen with the biomarker detected, the biomarker's role, personal caries' risk level, easy home recommendations, oral-systemic connections and how caries develops;

### DETAILED DESCRIPTION OF THE PREFERRED EMBODIMENT

In the following description, various operating parameters and components are described for different constructed embodiments. These specific parameters and components are included as examples and are not meant to be limiting. Unless otherwise noted, all technical and scientific terms used herein are to be accorded their common meanings as would be understood by one having ordinary skill in the art.

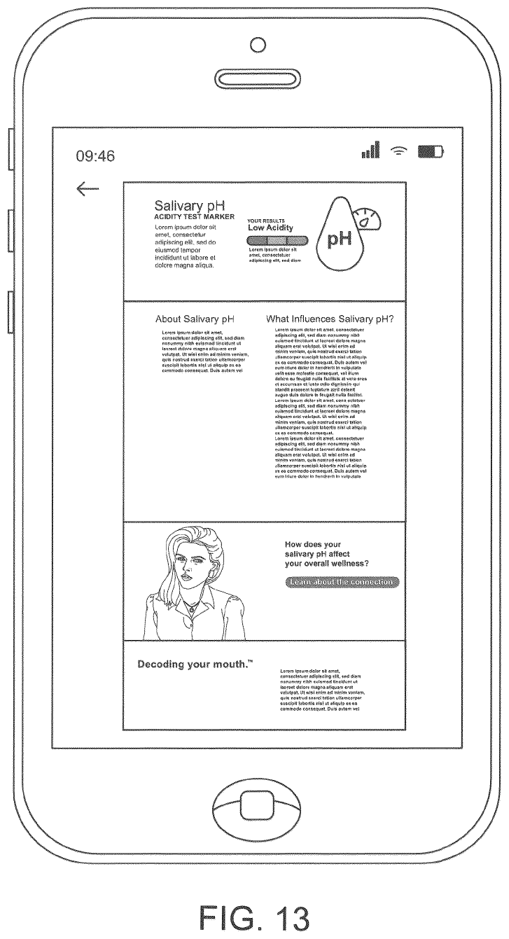

Known techniques for analyzing saliva conditions involve in-office saliva collection, timely mailing of specimen and laboratory technicians. Conversely, the disclosed inventive apparatus and method involves the use of the above-referenced SPIT test to simplify saliva testing and allow for real-time results with no human laboratory technicians. Processing of data and results are handled by computer vision automatically and result detection is based on a computer algorithm trained and continually refined as more tests are delivered. Applicant's SPIT design allows patients to obtain tangible results immediately, and also allows providers to make well-informed treatment decisions immediately and without the need to wait for laboratory results, revisiting health records and calling or scheduling the patient for another appointment to discuss prevention and treatment plans. The SPIT test's automated results also provide easy-to-understand reports with expandable details for patients to take action in their homes for better long-term health (FIG. 13).

The SPIT design test utilizes lateral flow assays and colorimetric assays to detect dental-specific biomarkers related to caries, periodontal disease, halitosis, Sjögren's Syndrome and candida. These markers include Glucosyltransferase (an enzyme produced by cariogenic bacteria), dipeptidyl peptidase-4/CD26 (serine protease overexpressed in Sjögren's Syndrome) and Atopobium parvulum, Eubacterium sulci, Fusobacterium periodonticum and Solobacterium moorei (halitosis-causing bacteria) and glucose.

These biomarkers not only determine oral health risk levels and provide at-home recommendations, but also help assist providers in long-term treatment decision modalities, safety and efficacy. Patients with high glucose levels indicate diabetes and lower ability to resist infection and slow healing. The SPIT design test allows providers to have a quick comprehensive picture of overall health for optimal treatment decisions.

Current salivary testing involves laboratory techniques such as Polymerase Chain Reactions (PCR) and Enzyme-Linked Immunoassays (ELISA). The SPIT design utilizes LFA and colorimetrics, laboratory technology that is known to be sensitive, highly successful for rapid analysis and inexpensive.

The SPIT design test utilizes several LFAs and colorimetrics. Applicant separated the assays with defined borders so there is no cross-reaction, utilizing buffering solutions to ensure salivary flowability and inserted into a single test card for ease of use.

### Method Steps—Collection and Computer Analysis

Applicant's system utilizes computer vision and machine learning techniques to analyze the biomarkers on the SPIT test and leverages specially trained computer vision models running on modern cloud infrastructure.

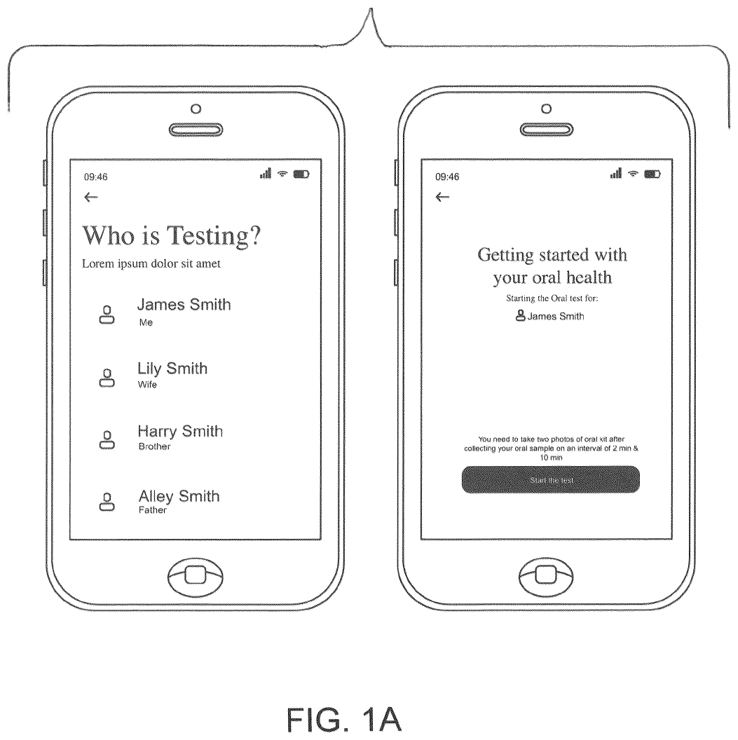

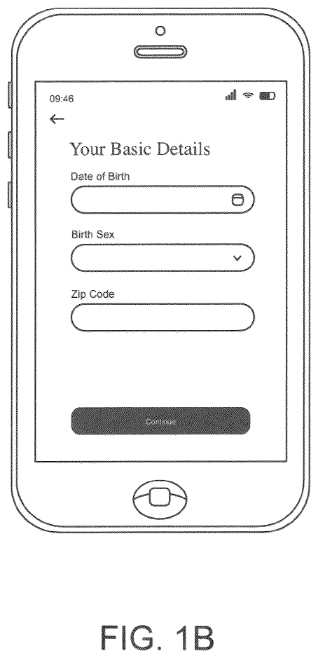

A robust Web application provided specifically for use with the disclosed apparatus and method for receiving patient information. An example of such a screen ready to receive patient information is illustrated in FIGS. 1A and 1B which are non-limiting samples. Input information may include, but may not be limited to, the patient's identification and test information. Once the patient information is inputted into the Web application, a Caries Management by Risk Assessment (CAMBRA) form also appears, asking questions related to risk factors including diet, oral hygiene, fluoride and past oral health history. The CAMBRA system takes into account caries disease indicators such as socio-economic status, developmental problems, and presence of lesions or restorations placed within the previous three years.

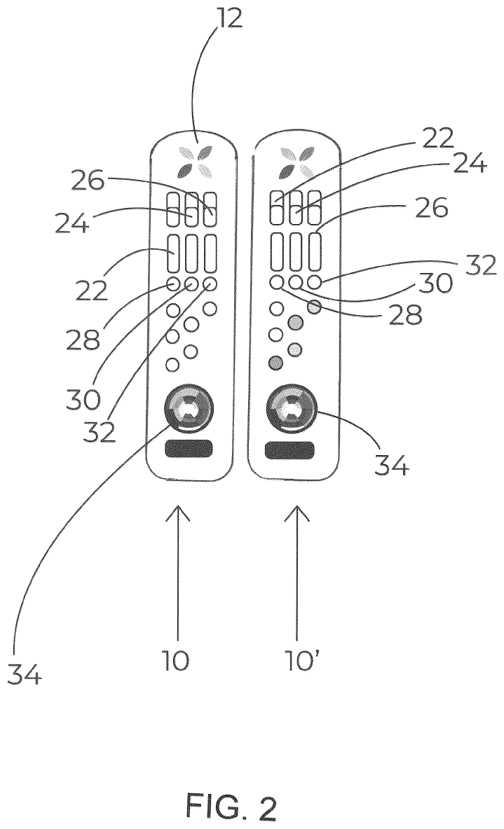

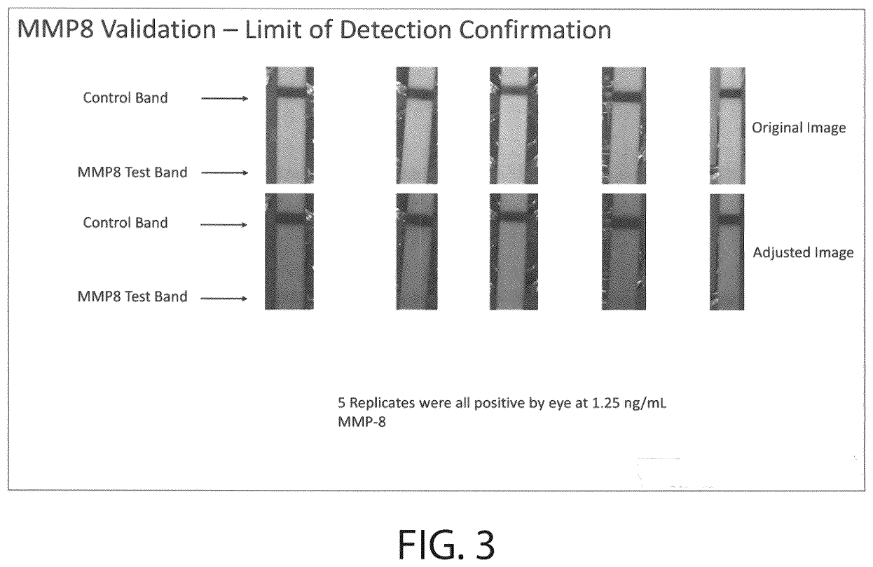

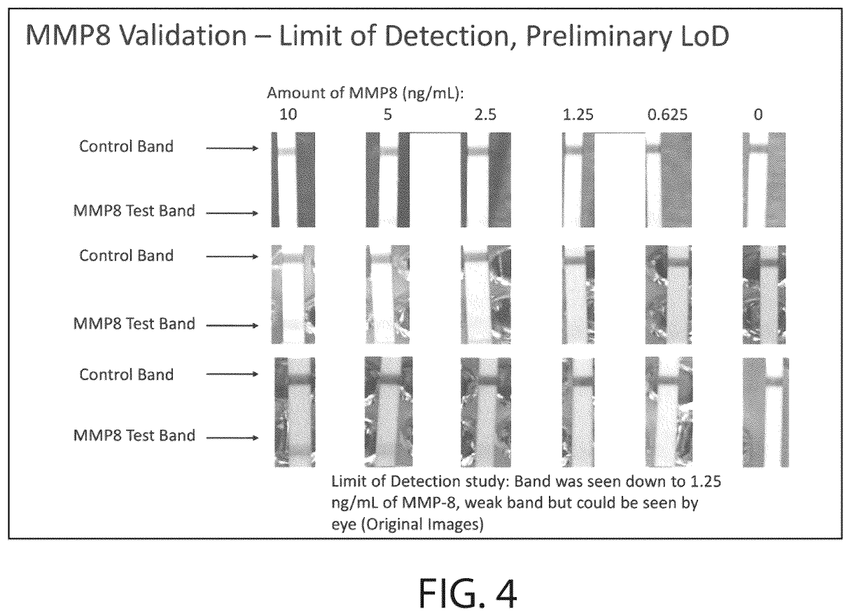

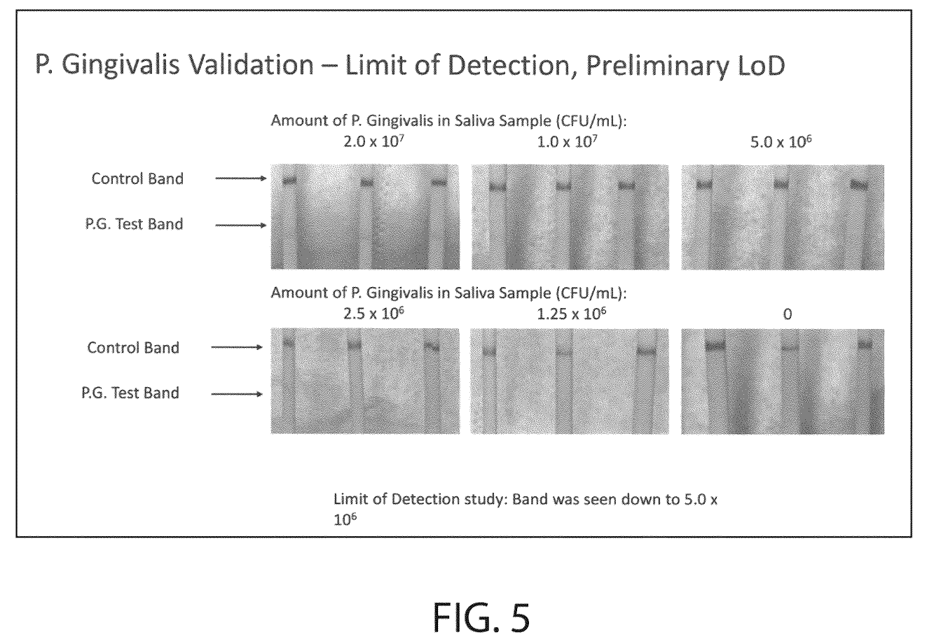

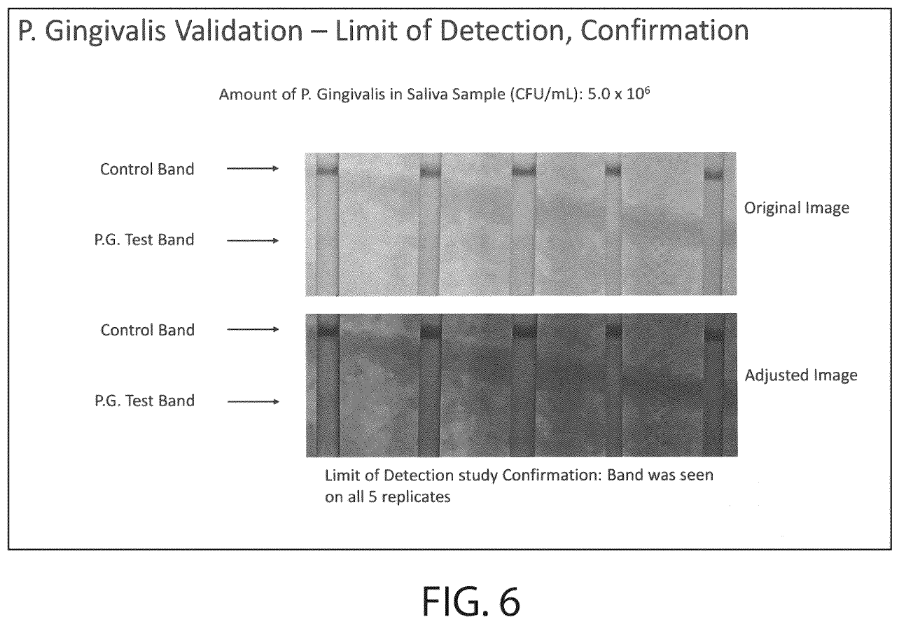

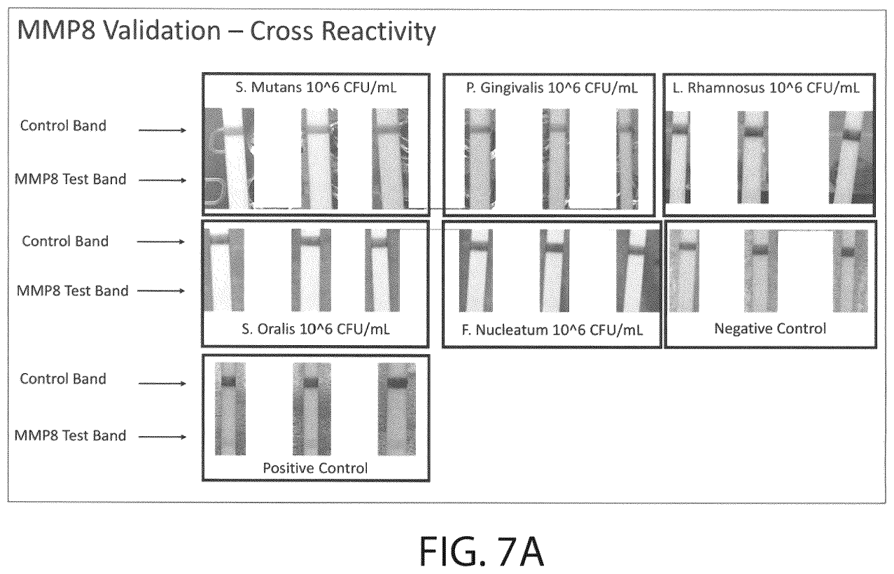

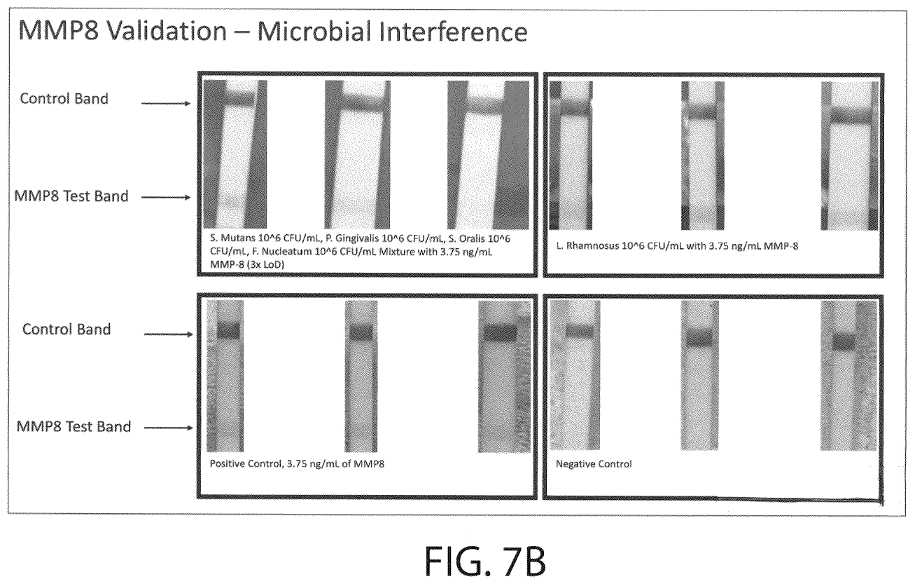

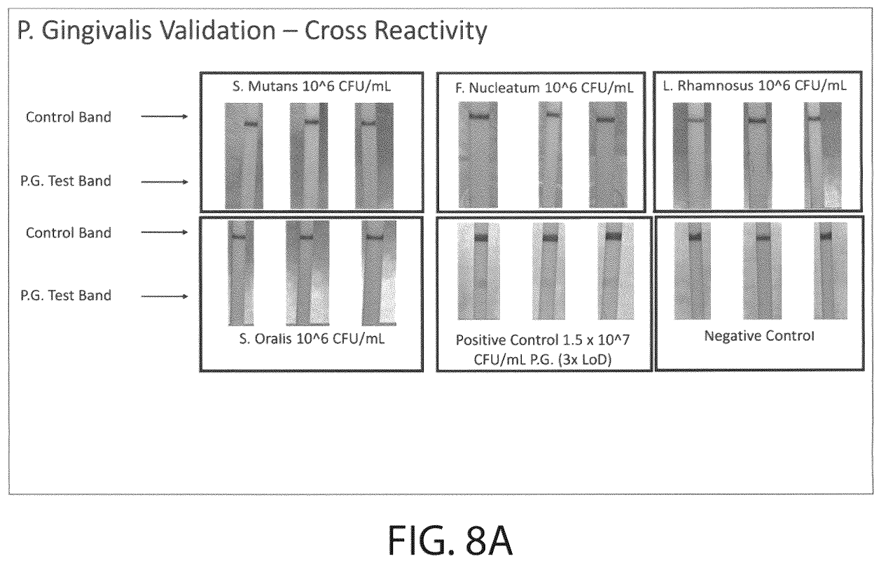

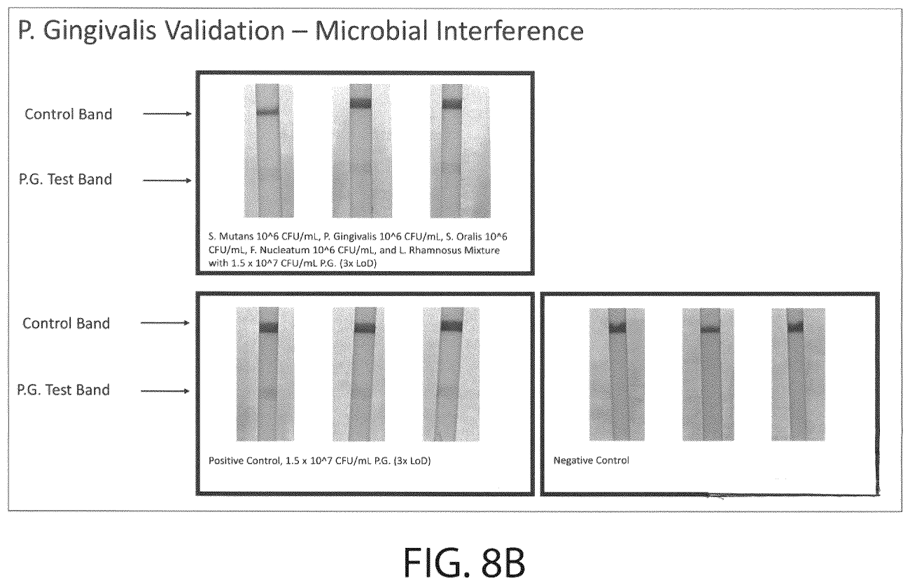

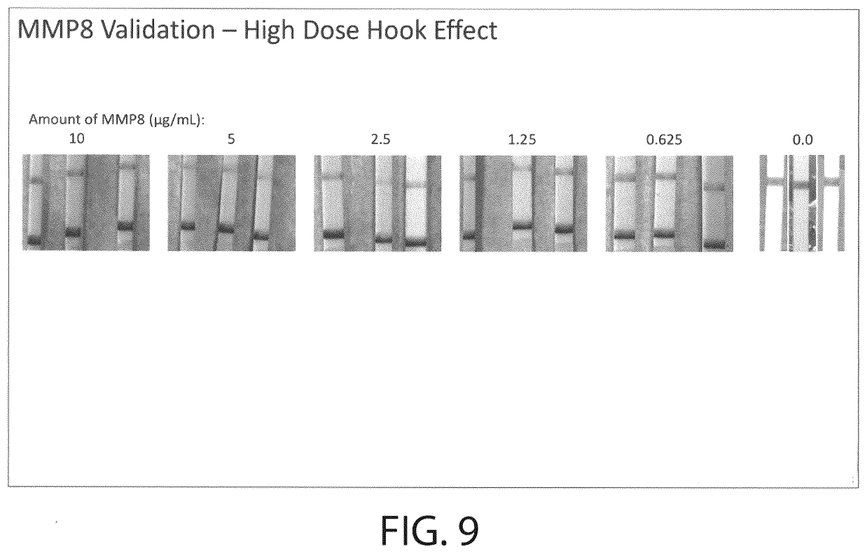

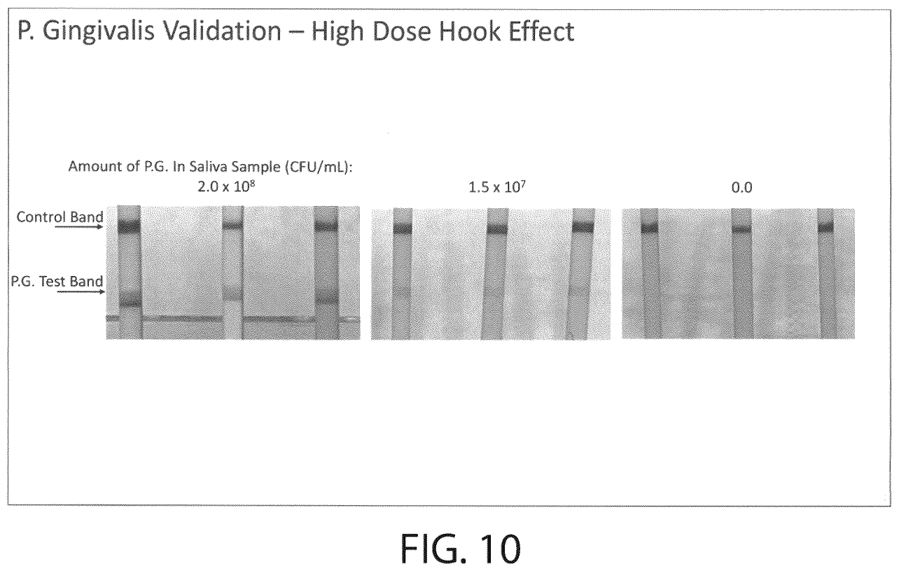

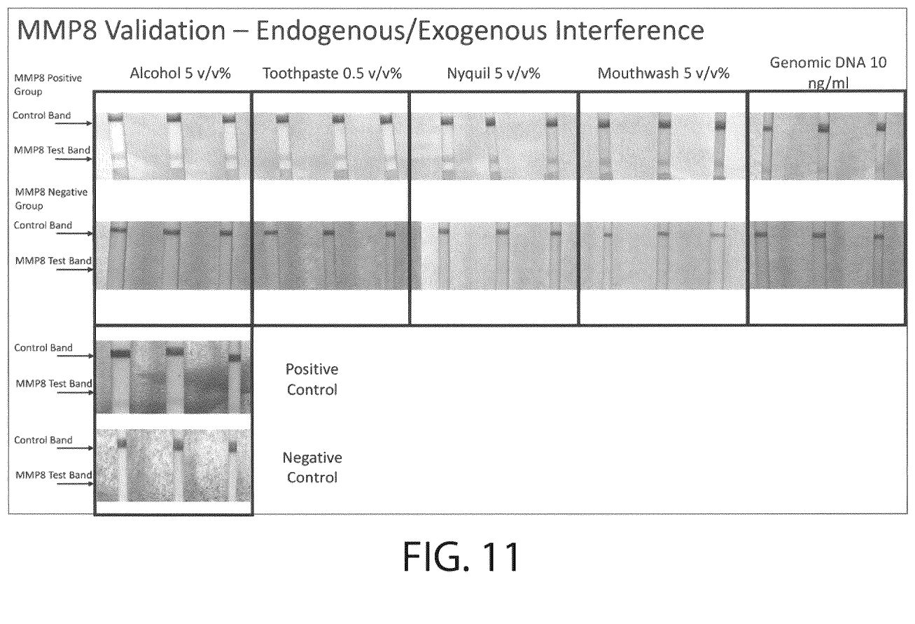

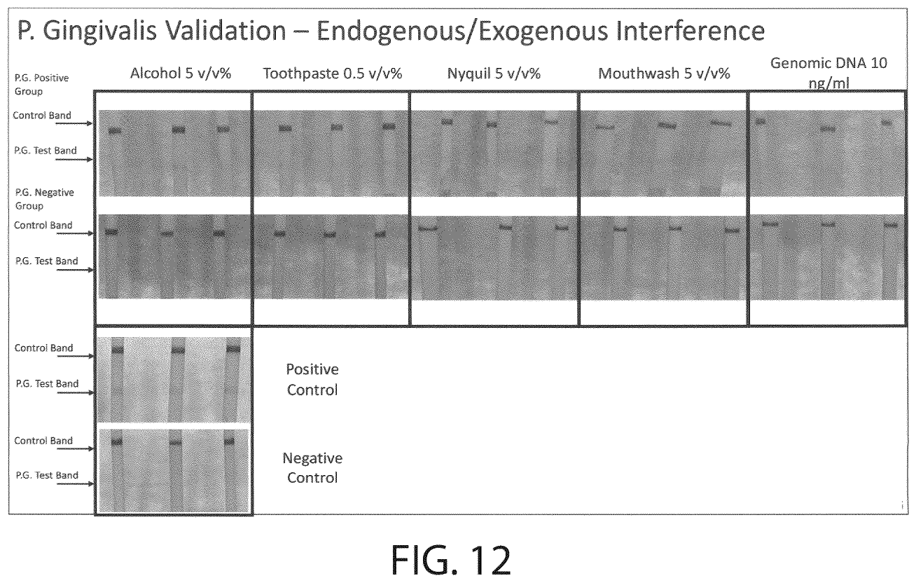

Once the patient information has been uploaded, the patient prepares for the test and uses the test cards illustrated in FIG. 2. As illustrated, a test card 10 is shown prior to testing while a test card 10′ is shown following testing. The test cards 10 and 10′ internally house of a plurality of immunoassay test strips and colorimetric/chemistry pads 22, 24, and 26 which can be partially visualized through windows formed in the body of the test card. A greater or lesser number of immunoassay test strips can be included. In addition to the windows, an equal number of lanes 28, 30, and 32 are formed in the cover of the test card 10.

The test card 10 has distinctive landmarks such as a company name or logo 12 that the computer vision detects. These landmarks help align the test and detect the intensity of the colors in correspondence to a provided color wheel reference 34. For example, a dark red band on the SPIT test may indicate a higher bacterial level, indicating a high likelihood of periodontal disease. The results detected then trigger an automated output of results and treatment considerations for patients and providers, along with which particular systemic conditions the biomarker affects. In addition to the test card 10, the test kit further includes a test kit box, a sponge having indicator bands, a transfer pipette, a main compression tube, three capped dropper vials each of which being a different color, and a starter guide having a QR code, none of which are shown. For purposes of illustration, the vial colors are purple, green and pink, though any colors may be used. The kit further includes a color comparison guide in which a key disease state key is included.

For the apparatus and method of the disclosed invention to operate at its maximum level of efficiency, the patient should refrain from eating or drinking for at least 30 minutes followed by rinsing the mouth with water for 30 seconds before administering the test. To administer the test, the patient scans the QR code on the starter guide to download the app.

Once the appropriate app is downloaded and the patient is prepared for the test, the following steps are taken by the patient.

First, the lot number on the side of the box is entered in the app.

Second, the three holes provided in the box are perforated to form stands to hold the collection vials. The cap of the purple-capped dropper vial is opened and the vial is placed in one of the perforated holes. The green and pink collection vials are then unsealed by removal of the caps and the foil seals and are then placed in their respective perforated holes.

Third, the patient washes his or her hands thoroughly with soap and water.

Fourth, saliva is allowed to pool in the patient's mouth for one minute.

Fifth, the compression tube is removed and set aside.

Sixth, the patient gently places only the tip of sponge in his or her mouth and allows saliva to be absorbed into sponge until the area above the bands turns a different color, a step which may take up to three minutes.

Seventh, with the sponge pointing upwards, the patient places the compression tube over the sponge.

Eighth, the compression tube/sponge combination is inverted and tip of the sponge is place into the purple capped dropper vial. The compression tube is then pressed down completely to compress sponge to thereby dispense saliva into the purple capped dropper vial.

Ninth, the compression tube is removed from dropper vial. Thereafter, the purple dropper cap is placed onto the dropper vial and the cap is removed from the dropper tip.

Tenth, the dropper bottle is squeezed to dispense one drop of saliva through dropper bottle tip into the green collection vial while three drops of saliva are dispensed through the dropper bottle tip into the pink collection vial.

Eleventh, the caps on the green and pink collection vials are securely closed. The vials are then inverted ten times.

Twelfth, three drops of saliva are dispensed from the green collection vial into lane 34 of the card 20 and three drops of saliva from the pink collection vial are dispensed into lane 36.

At this point, the mobile app asks “Have you completed these steps?” and, if completed, the patient clicks “check box.” A timer counts for eight minutes on the mobile app and then instructs the patient to take the “ADA Caries Risk Assessment and ADA Diabetes Risk Test.”

After six minutes, the following instructions appear on the screen: (1) Unscrew the purple cap of the dropper vial of collected saliva specimen, (2) use the transfer pipette provided in the kit to pipette saliva from the dropper vial, (3) and one drop of saliva is placed into each of the colorimetric wells.

The mobile app then asks “Have you completed these steps?” Click “Checkbox” and, if completed, the patient clicks “check box.”

The timer counts out two minutes after which a button appears to guide the patient to take photo of the test card which is then uploaded to the app for analysis.

The final analysis appears on the patient's computer screen as illustrated in FIG. 13. As illustrated, in this instance the patient's saliva demonstrated a low pH, a satisfactory result. Details regarding this condition are discussed, however, any dental condition could be assessed and discussed. The illustrated information card is intended as being suggestive and not limiting.

A cloud-based AI and machine learning computer vision technology was developed which transforms data into actionable healthcare decisions. An algorithm to support the SPIT test and results was created. The technology is adaptable for use in areas where reception is limited, thereby assisting underserved populations.

### Unique Characteristics

The SPIT test involves the healthcare provider and patient. Healthcare providers can include dentists, dental hygienists, dental assistants, physicians, physician assistants, nurses and nursing assistants. These healthcare providers can be in dental offices, nursing homes, community clinics and more. The easy, non-invasive methods of saliva collection and result detection also allow providers to send test kits to patient homes for remote testing, monitoring and care decisions.

Throughout the entire workflow, the healthcare provider and/or patient are guided through the process in the application with step-by-step videos. These videos significantly reduce the amount of user error to ensure accuracy of results. Furthermore, the application guides the user's hands when taking the test card photo. If the image has too much glare, is angled incorrectly or captured in incorrect lighting or background, the application immediately notifies the user to rescan and reimage the test card. Limited human technical support is needed to gather data, allowing the disclosed apparatus and method to be more scalable and the test to be taken anywhere, at any time and by anyone regardless of healthcare experience.

Once results are analyzed through computer vision and machine learning techniques, a provider views the report and makes home recommendations and modifications, if need be, based on dexterity, health issues and other possible impacting factors. The provider can also utilize the results in conjunction with the clinical exam to evaluate other caries risk factors such as visible accumulations of plaque, fissure anatomy, root surface exposure and presence of appliances and periodontal disease risk factors such as pocket depth, mucogingival effect, recession and bleeding on probing. Sjögren's disease risk can also be evaluated to help in long-term care and confirm diagnosis as current methods for detection are only visually seen as dry eyes and dry mouth.

Treatment decisions can also be made for better long-term planning and care management. For example, those with high glucose levels tend to experience reduction of nitric oxide secretion, leading to poor circulation and slow-healing sockets. Dentists can avoid severe dental treatment complications when glucose levels at time of treatment are considered.

The development of the SPIT test and easy use by anyone also allows for remote monitoring. The slimness of the test cards paired with a commonly found household device allows anybody to monitor their dental health anywhere. Results can be monitored and managed by providers in clinics, and those with elevated levels of certain biomarkers can be brought in for necessary in-office treatment.

The lack of objective and non-invasive dental testing and monitoring with imaging diagnostics to accelerate clinical implantation of reliable, reproducible, highly specific and sensitive diagnostic instruments for dental caries, periodontal disease and Sjögren's Syndrome is a well-defined problem. Current methods to detect and predict the progression of dental caries, periodontal disease, reversible and irreversible pulpitis need to be improved and fully resolved.

The development of the SPIT test with computer vision and machine learning is critical to advance and elevate patient care standards. Following the development of the test cards and computer vision according to the disclosed apparatus and method, the rest results were validated with patients having known caries, periodontal disease, halitosis, diabetes and Sjögren's Syndrome to ensure clinical correlation. The validations ensure real-world performance, understandable instructions for use (IFU), and accuracy.

In summary, the disclosed apparatus and method provides an automated salivary test analysis utilizing computer vision and machine learning techniques, as well as providing a better comprehensive visual for providers to make the best clinical treatment decisions. The disclosed apparatus and method increases scalability while enabling patients and providers to obtain the full picture of their oral health, how to better prevent oral diseases and how it relates to their systemic health.

Almost half of the world's population suffers from oral diseases. Untreated tooth decay affects 2.5 billion people. Complete tooth loss affects 350 million people. Severe gum disease affects 1 billion people. Oral cancer affects 380,000 people [World Health Organization, 2022]. The disclosed apparatus and method provides the information providers and patients need to better reduce these numbers and help people globally achieve better health.

### Study Background

Applicant determined the feasibility of MMP-8 and P. gingivalis. Applicant tested various antibodies for detection ELISA by performing serial dilutions of the antibodies to determine which one had the highest sensitivity. Applicant also tested for specificity and tested against multiple related antigens to show that the antibody was highly specific to the markers and no false positives were generated due to presence of other commonly found substances in saliva. Applicant took the most ideal screened antibody and developed detection through the LFA.

Applicant performed the same procedural methods for Streptococcus mutans, a commonly known cavity-causing bacteria. Although Applicant successfully detected corresponding antibodies on the ELISA and had custom developed an antibody for Glucosyltransferase (GTF), an enzyme that is produced by all cariogenic bacteria. In effect, measuring GTF measures the level and presence of all cariogenic bacteria beyond S. mutans [International Journal of Oral Science, 2021]. Thus, the development of a custom antibody for GTF allows for its detection on a LFA for caries risk assessment.

Applicant prepared buffering solutions to ensure flowability of the saliva as salivary viscosity varies between individuals. Applicant's running buffering solution for MMP-8 consists of 1× phosphate buffered saline (PBS), 1% Ten-20, and 2% bovine serum albumin (BSA). Applicant's running buffering solution for P. gingivalis consists of 50 nM boric acid (pH 9.0), 1% Ten-20, and 2% Bovine Serum Albumin (BSA). This research resulted in the development of one universal running buffering solution for all markers to simplify use and prevent any mix-ups.

### Initial Steps

Following the completion of an assay feasibility study, Applicant completed assay development and assay production. Further development resulted in optimization and the development of the two sandwich immunoassays and scaled production. Included in those efforts was the development of calibrators and controls (in-process, final release) needed for reliable and reproducible manufacture of the lateral flow assays. A custom card was designed to house the immunoassays and complimentary chemistry assays. In addition, multiple assays were developed for evaluation to ensure usability and accuracy.

Applicant formulated one running buffering solution that universally works for all biomarkers and screened and tested known antibodies for Glucosyltransferase (an enzyme produced by cariogenic bacteria), dipeptidyl peptidase-4/CD26 (serine protease overexpressed in Sjögren's Syndrome) and Atopobium parvulum, Eubacterium sulci, Fusobacterium periodonticum and Solobacterium moorei (halitosis-causing bacteria).

Throughout optimization and development, Applicant used various image-capturing devices such as Apple iPads, Android tablets, Apple smartphones and Android smartphones to capture the LFA bands and colorimetric assay colors over time and with multiple saliva samples. Capturing these images assisted in preliminary training of the Web application and computer vision and machine learning aspects.

The steps outlined above resulted in the development of prototypes and production models for use in apparatus demonstration models for healthcare insurances such as Medicare Advantage and Medi-caid and dental clinics to potentially create business value and better patient care.

### Optimization, Development, and Scaling Up For Manufacturing

Overall Strategy: During Applicant's prior work, Applicant completed assay feasibility for MMP-8, P. gingivalis, pH and buffering capacity. Applicant next converted these results into a work plan to optimize, develop and transfer the assays. Applicant developed calibrators and controls needed for reliable and reproducible manufacturing of LFAs, and completed assay optimization and development.

Methodology & Analyses: Based on the test strips that have been shown functional for MMP-8 and P. gingivalis, Applicant completed the Optimization and the Development of the two point of care tests. These efforts included but were not limited to antibody conjugation to detector/label optimization, optimizing the antibody concentrations and dispensing buffer compositions for the capture reagents, optimizing and developing dispensing methods, buffers and drying times; in addition, work to finalize the running/drying buffers for the two assays was performed by the development scientists. The final assay workflow (e.g. sample treatment; sample and wash buffer volumes; sequence of additions; incubation times; read times, etc.) confirmed prior to a thorough performance evaluation of both, the MMP-8 and the P. gingivalis assays. Performance studies included contrived saliva samples to determine the Limit of Detection (LoD), cross-reactivity and interfering substances testing as well as Flex studies to determine the robustness of the test strips. In addition, sample correlation with both positive and negative, characterized samples were included in the testing. Stability studies for key assay components and full test strips (accelerated and real time) established the product shelf life. Stabilizers may be added to extend shelf life if needed.

Alternative Strategies: Applicant's initial studies have already shown functional prototype assays. During this phase of the development, Applicant addressed specificity challenges (i.e. non-specific binding) by adding protein blockers (e.g. BSA, casein, etc.), surfactants (e.g. Ten-20, Brij-35, TritonX100, Surfactant 10G, Tetronic, Pluronic, etc.), polymers, salts, etc. to the running buffer to minimize and eliminate these issues. For persistent challenges, Applicant also included changes in buffer types, ionic strengths, and pH.

### Creation of Universal Running Buffering Solutions

Overall Strategy: To simplify testing and ensure straightforward ease of use, Applicant developed a universal running buffering solution that can be utilized for all oral biomarkers. Applicant's prior work has two separate running buffering solutions, which can create complications if solutions are accidentally mixed and dispensed into non-corresponding wells. Utilizing only one running buffering solution that can be utilized for all assays simplifies the process.

Applicant tested known antibodies for Glucosyltransferase (an enzyme produced by cariogenic bacteria), dipeptidyl peptidase-4/CD26 (serine protease overexpressed in Sjögren's Syndrome) and Atopobium parvulum, Eubacterium sulci, Fusobacterium periodonticum and Solobacterium moorei (halitosis-causing bacteria) and glucose.

Methodology & Analyses: This process involved the development of new antibodies with specific characteristics. Possible changes that can be made are the gold nanoparticle size, buffer used, pH, concentration. Other possible changes considered include the membrane used, antibodies (as mentioned above), labels/particles, conjugate pad, and chemistry of reagent used.

Alternative Strategies: An alternative strategy that Applicant considered was to apply the different buffer directly onto the LFA and reagent test themselves. This method accomplishes the same desired result as developing a universal running buffering solution.

### Use of Robust Web Application to Detect Test Results

Overall Strategy: With recent advances in computer vision, machine learning, and image processing, Applicant developed a system which can consume an image of the developed and optimized assays in order to analyze and provide bespoke recommendations in near real-time. Applicant's system analyzes the test card image position, color, and intensity of the LFA bands and colorimetric assays, removing the need for a human laboratory technician.

Methodology & Analyses: The computer vision is programmed with a set parameter of the layout of the test kit along with a color chart to calibrate the external lighting of the image taken. Using the set layout of the test kit, the computer vision identifies specific locations required to “read” the color. After the color is read, a numerical value correlated to the color and intensity or brightness is assigned to the specific location. The numerical value is then referenced to a predetermined set of values that correlates to low, medium or high for a specific biomarker. The computer vision uses the location of the color to determine which biomarker is indicated, and uses the numerical value to determine if the reading is assigned as a low, medium or high indication.

Alternative Strategies: Alternative strategies involve the use of a technician to help train the computer vision. With Applicant's stored database of the images of each test that is uploaded to the Web application, a technician can check the accuracy of the computer vision.

### Further Studies—In General

As a next step, Applicant prepared the assay prototypes and placed them in a manufacturable test card and card as illustrated in FIGS. 1A and 1B and as discussed in relation thereto. Applicant also trained the web application's computer vision and machine learning on the final products to ensure accuracy and ease of use. Applicant scientifically validated the test results and computer vision application with spiked saliva substitute of the biomarkers discussed above at varying levels.

### Calibration and Standardization

Overall Strategy: Successful manufacturing depended on thorough assay and process development, and the establishment of calibrators and standards for product consistency over time. Applicant selected and established the relevant gold, silver and working standards for both assays. Commercially available recombinant MMP-8 were selected as standards/controls for the second assay. Applicant screened various reagents from different sources and selected and validated the relevant materials for gold standards. Relevant levels of the standards/calibrators were determined for each of the assay based on clinical utility and assay performance.

### Methodology & Analyses:

Scale-up: during scale up from small batches (200-500 tests) to medium sized batches (3,000-5,000 tests) to large production size batches (>20,000 tests) various lots of critical materials and reagents, ideally three different batches, were tested. Materials included nitrocellulose membrane, antibodies, antigens (gold standards and working standards; calibrators, controls), labels/particles, conjugate and sample pad, key reagents, and critical assay accessories. Using those materials, the following processes were deemed critical and were scaled up to production size levels: Antigen-establish gold standard(s), in-process and final QC controls and standards, Label conjugations, Dispensing processes (membrane and conjugate, and others if needed), Drying processes (membrane and conjugate, and others if needed), Develop standard curves as needed, Process Validation Plan and Protocol and Process Validation Report.

Alternative Strategies: During the studies, Applicant found that it is possible that some scale-up efforts require additional efforts to produce consistent intermediate products. For example, the reaction and blocking times for conjugates might need adjustment, and the drying times for membranes, conjugate and sample pads (if needed) might have to be increased for larger batches. However, the expected challenges will be minor and can readily be addressed by process updates.

### Refinement of Web Application's Computer Vision and Machine Learning

Overall Strategy: While the Web application's computer vision and machine learning were trained early in Applicant's research, further refining of the algorithm and process in the final manufacture of card and card products used in the present method will be possible taking into consideration the additional markings and guides built into the test cards to ensure accurate detection.

Methodology & Analyses: As noted above, using the manufactured test cards Applicant took the images on various mobile tablet and phone devices and trained the program on identifying the markings, white spaces and lighting considerations for detection. Applicant confirmed the results are correct utilizing the human eye.

Alternative Strategies: Applicant's system uses past images from prototypes and new images from the final finished test card to train the computer vision. As an alternative to program training, Applicant is able to manually analyze the test cards, capture and store the images and build a larger library of images as required to get successful computer vision training completed.

### Validation and Correlation

Overall Strategy: Following the development of the test cards, Applicant scientifically validated the test results with spiked saliva substitute for all the biomarkers at varying concentrations to simulate patients with caries, periodontal disease, halitosis, diabetes and Sjögren's Syndrome. The results demonstrated that the test assays were accurate following known principles. Applicant utilized the computer vision application to image the test cards with the spiked saliva substitute on various imaging devices (i.e. iPad, tablet, smartphone).

Methodology & Analyses: Saliva substitute were spiked with varying concentrations of all biomarkers to equivalate a high risk, moderate risk and low risk for each of the categories—Caries, Periodontal Disease, Halitosis, Diabetes and Sjögren's Syndrome. The study simulated 10 expected high risk, 10 expected moderate risk and 10 expected low risk tests, yielding a total of 150 tests. Applicant took the captured results and checked the corresponding biomarkers displayed on the test results match the clinical condition. These images were repeated on three different imaging devices. These recordings were documented into a final scientific validation study.

Alternative Strategies: The greatest risk to this aim was unmatching results, which may result in revisiting either assay calibration or computer vision calibration. In that case, Applicant would have had to first look at computer vision calibration and have a trained human manually read the test results from the collected test card image. If the results were correct, then Applicant would have taken a deeper look into the assays to determine if adjustments to sensitivity were needed.

I claim:

1. A method of testing a saliva sample to determine the oral health parameters of a patient, comprising:
forming a saliva test kit including a test card, the test card having at least one lateral flow assay and at least one colorimetric assay, the assays being separated on the test card, the assays detect dental specific biomarkers related to dental caries, periodontal disease, halitosis, Sjögren's Syndrome and candida, the card further including apertures for placement saliva, test strip visualization, and color visualization;
downloading a saliva test application onto a mobile device;
opening a patient file on the mobile device using the saliva test application;
collecting a volume of the patient's saliva;
placing an amount of the patient's saliva on the saliva aperture of the test card;
allowing time for the assays to react to the patient's saliva;
confirming that at least one band has appeared on the test strip and that a color change has taken place;
capturing the image of the test card on a mobile device following the appearance of the at least one band and the color change;
uploading the captured image of the test card into the application; and
reading or having read the results of the test.

2. The method of claim 1, wherein the biomarkers are selected from the group consisting of Glucosyltransferase, a dipeptidyl peptidase. Atopobium parvulum, Eubacterium sulci, Fusobacterium periodonticum and Solobacterium moorei.

3. The method of claim 2, wherein the dipeptidyl peptidase is dipeptidyl peptidase-4/CD26.

4. The method of claim 2, wherein the dipeptidyl peptidase is the serine protease overexpressed in Sjögren's Syndrome.

5. The method of claim 1, wherein the lateral flow assay and colorimetric assay comprise a plurality of immunoassay test strips.

6. The method of claim 5, wherein the test card includes a top side wherein the plurality of apertures for placement saliva, test strip visualization, and color visualization are formed.

7. The method of claim 6, wherein the top side of the test card further includes a color wheel against which the color of the color indicator may be compared.

8. The method of claim 7, further including a color key for identifying a possible disease state.

9. The method of claim 1, wherein the assays are two sandwich immunoassays.

9. The method of claim 8, including the step of comparing the color of the color indicator against the color wheel to identify a color change and identifying the disease state by reviewing the color key.

10. The method of claim 1, further including a buffering solution for use in adjusting saliva viscosity for use with the test card.

11. The method of claim 10, wherein the buffering solution is compatible with all of the biomarkers.

12. The method of claim 10, wherein the buffering solution consists of 1× phosphate buffered saline and 1% Ten-20, and 2% bovine serum albumin.

13. The method of claim 10, wherein the buffering solution consists of 50 nM boric acid (pH 9.0) and 1% Ten-20, and 2% Bovine Serum Albumin.

14. A test kit for testing the saliva sample of a patient to determine the oral health of a patient, the test kit including a test card having plural immunoassay strips for visually responding to the presence of a biomarker, the kit further including a buffering solution compatible with all biomarkers related to dental caries, periodontal disease, halitosis, Sjögren's Syndrome and candida.

15. The test kit of claim 14, wherein the solution is screened for specific antibodies including antibodies for Glucosyltransferase, dipeptidyl peptidase, Atopobium parvulum, Eubacterium sulci, Fusobacterium periodonticum and Solobacterium moorei.

16. The test kit of claim 15, wherein the dipeptidyl peptidase is dipeptidyl peptidase-4/CD26.

17. The test kit of claim 16, wherein the dipeptidyl peptidase is the serine protease overexpressed in Sjögren's Syndrome.

18. The test kit of claim 14, wherein the buffering solution consists of 1× phosphate buffered saline (PBS) and 1% Ten-20, and 2% bovine serum albumin

19. The test kit of claim 14, wherein the buffering solution consists of 50 nM boric acid (pH 9.0) and 1% Ten-20, and 2% Bovine Serum Albumin.

20. The test kit of claim 14, wherein the test card includes a front side, the front side including apertures for placement saliva, test strip visualization, and color visualization, the front side further including a color wheel.
